(19) United States
(12) Patent Application Publication
(10) Pub. No.: US 2005/0059906 A1
(43) Pub. Date: Mar. 17, 2005

(54) Specimen collector

(76) Inventor: Garry Tsaur, Rowland Heights, CA (US)

Correspondence Address: JOE NIEH, 18760 E. AMAR ROAD #204, WALNUT, CA, 91789, US

(21) Appl. No.: 10/661,997

(22) Filed: Sep. 12, 2003

(51) Int. Cl. A61B 5/00

(52) U.S. Cl. 600/573

(57) ABSTRACT

An economical specimen collector comprising a hollow elongated tubular housing with a sealed end and an open end with an opening means provided near the sealed end. A viscous substance may also be disposed near the open end of the hollow elongated tubular housing to seal the collected specimen in the hollow elongated tubular housing. The specimen collector may be used to collect liquid specimen and subsequently release the specimen by allowing air to enter the hollow elongated tubular housing through the opening means.

### BACKGROUND-FIELD OF INVENTION

The present invention relates generally to a device for collecting specimen. More specifically the present invention relates to an economical specimen collector for collecting liquid specimen.

### BACKGROUND-DESCRIPTION OF RELATED ART

Specimen often needs to be collected for examination and testing from a subject. A technician or the subject himself may perform the collection of specimen. The specimen may be collected by using a spatula to obtain the specimen and then placing the specimen in a separate container. The specimen may also be collected by using a vacuum container with preexisting vacuum in the container to suck the specimen into the container to be collected. However, the vacuum container has a limited shelf life due to the slow leakage of air into the vacuum container. Furthermore, a vacuum container generally has a relatively large volume to allow a reasonable amount of specimen to be collected even after some reduction of vacuum due to the leakage of air into the vacuum container.

### SUMMARY OF THE INVENTION

The present invention is an economical specimen collector comprising a hollow elongated tubular housing with a sealed end and an open end with an opening means provided near the sealed end. A viscous substance such as silicone may also be disposed near the open end of the hollow elongated tubular housing to seal the collected specimen in the hollow elongated tubular housing. The specimen collector may be used to collect liquid specimen and subsequently release the specimen by allowing air to enter the hollow elongated tubular housing through the opening means.

### BRIEF DESCRIPTION OF THE DRAWINGS

FIG. 1 shows the preferred embodiment of the specimen collector.

FIG. 2 shows another embodiment of the specimen collector.

### DETAILED DESCRIPTION OF THE PREFERRED EMBODIMENT

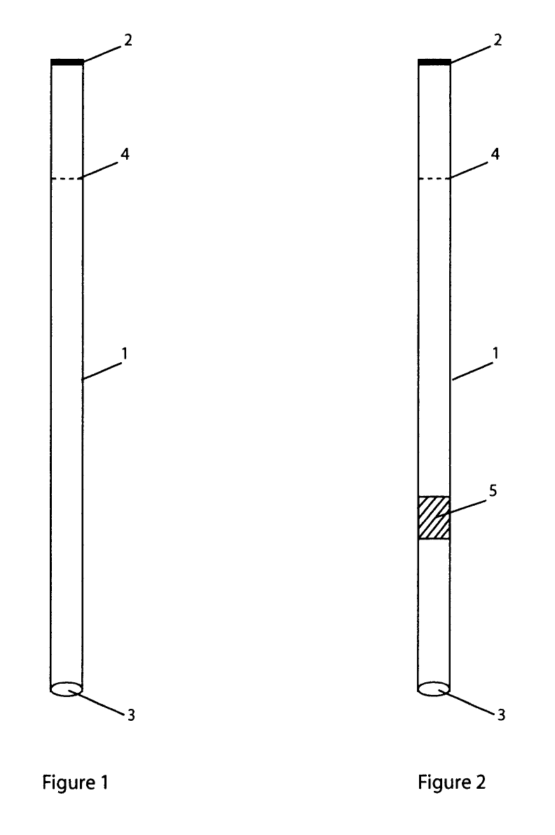

FIG. 1 shows the preferred embodiment of the present invention. In the preferred embodiment, the specimen collector comprises of a hollow elongated tubular housing 1 with a sealed end 2 and an open end 3 with an opening means 4 provided near the sealed end 2. The preferred opening means 4 is a score line that will allow the hollow elongated tubular housing 1 to be broken open at the score line. Other suitable opening means such as a snap-on cap or a screw-on cap may also be used. The specimen collector may be used to collect liquid specimen and subsequently release the specimen by allowing air to enter the hollow elongated tubular housing 1 through the opening means 4.

The specimen collector may be used to collect liquid specimen such as saliva by placing the open end 3 of the specimen collector into a subject's mouth. The subject need merely first suck the air from the specimen collector by creating a negative pressure inside the subject's mouth cavity. The subject may then place the open end 3 of the specimen collector in contact with the subject's saliva and allow air to re-enter his mouth. The negative pressure in the specimen collector will then suck the subject's saliva into the hollow elongated tubular housing 1 of the specimen collector to be collected and removed. The collected specimen may subsequently be released for examination and testing by simply allowing air to enter the specimen collector near the sealed end 2 through the opening means 4 to release the collected specimen through the open end 3 of the specimen collector.

FIG. 2 shows another embodiment of the present invention. In this embodiment, the specimen collector comprises of a hollow elongated tubular housing 1 with a sealed end 2 and an open end 3 with an opening means 4 provided near the sealed end 2 and a viscous fluid 5 provided near the open end 3. The preferred opening means 4 is a score line that will allow the hollow elongated tubular housing 1 to be broken open at the score line. Other suitable opening means such as a snap-on cap or a screw-on cap may also be used. The preferred viscous fluid 5 is silicone but any other suitable viscous fluid may also be used. The specimen collector may be used to collect liquid specimen and subsequently release the specimen by allowing air to enter the hollow elongated tubular housing 1 through the opening means 4. The viscous fluid 5 will seal the collected liquid specimen in the hollow elongated tubular housing 1. Subsequently, when air is allowed to enter the hollow elongated tubular housing 1 through the opening means 4 the collected specimen will be released by exiting though the resulting opening forced through the viscous fluid 5 by the specimen.

The specimen collector may be used to collect liquid specimen such as saliva by placing the open end 3 of the specimen collector into a subject's mouth. The subject need merely first suck the air from the specimen collector by creating a negative pressure inside the subject's mouth cavity. An opening will be formed through the viscous fluid 5 due to the exiting of the air within the hollow elongated tubular housing 1. The subject may then place the open end of the specimen collector in contact with the subject's saliva and allow air to re-enter his mouth. The negative pressure in the specimen collector will then suck the subject's saliva into the hollow elongated tubular housing 1 of the specimen collector to be collected and removed. The viscous fluid 5 will reseal itself after the saliva enters the hollow elongated tubular housing 1 and equalized the pressure in the hollow elongated tubular housing 1 and the external atmosphere. The collected specimen may subsequently be released for examination and testing by simply allowing air to enter the specimen collector near the sealed end 2 through the opening means 4 to release the collected specimen through the viscous substance 5 and open end 3 of the specimen collector.

Although the description above contains many specificities, these should not be construed as limiting the scope of the invention but as merely providing illustrations of some of the presently preferred embodiments of this invention. Thus the scope of the invention should be determined by the appended claims and their legal equivalents, rather than by the examples given.

1. A specimen collector comprising a hollow elongated tubular housing with a sealed end and an open end with an opening means provided near said sealed end wherein the specimen collector may be used to collect liquid specimen and subsequently release the specimen by allowing air to enter the hollow elongated tubular housing through said opening means.

2. A specimen collector as in claim 1, wherein the opening means is a score line.

3. A specimen collector as in claim 1, wherein the opening means is a snap-on cap.

4. A specimen collector as in claim 1, wherein the opening means is a screw-on cap.

5. A specimen collector comprising a hollow elongated tubular housing with a sealed end and an open end with an opening means provided near said sealed end and a viscous fluid provided near said open end wherein the specimen collector may be used to collect liquid specimen and wherein said viscous fluid will seal the collected liquid specimen in the hollow elongated tubular housing and subsequently release the specimen by allowing air to enter the hollow elongated tubular housing through said opening means.

6. A specimen collector as in claim 5, wherein the opening means is a score line.

7. A specimen collector as in claim 5, wherein the opening means is a snap-on cap.

8. A specimen collector as in claim 5, wherein the opening means is a screw-on cap.

9. A specimen collector as in claim 5, wherein the viscous fluid is silicone.

10. A specimen collector as in claim 6, wherein the viscous fluid is silicone.

11. A specimen collector as in claim 7, wherein the viscous fluid is silicone.

12. A specimen collector as in claim 8, wherein the viscous fluid is silicone.
